(19) United States
(12) Patent Application Publication
(10) Pub. No.: US 2014/0212477 A1
(43) Pub. Date: Jul. 31, 2014

(54) HEMOGLOBIN-CONTAINING LIPOSOME AND METHOD FOR PRODUCING SAME

(71) Applicant: TERUMO KABUSHIKI KAISHA, Shibuya-ku (JP)

(72) Inventors: Shinichi KANEDA, Kanagawa (JP); Hiroshi Goto, Kanagawa (JP); Tsutomu Ueda, Kanagawa (JP); Takanobu Ishizuka, Kanagawa (JP); Shinji Motoyama, Kanagawa (JP)

(73) Assignee: TERUMO KABUSHIKI KAISHA, Shibuya-ku (JP)

(21) Appl. No.: 14/227,849

(22) Filed: Mar. 27, 2014

Related U.S. Application Data
(63) Continuation of application No. PCT/JP2012/073810, filed on Sep. 18, 2012.

(30) Foreign Application Priority Data
Sep. 28, 2011 (JP) 2011-213416

(51) Int. Cl. A61K 38/42; A61K 9/127

(52) U.S. Cl. CPC A61K 38/42; A61K 9/1271; A61K 9/1277
USPC 424/450; 514/13.5

(57) ABSTRACT

Provided are a hemoglobin-containing liposome having a specific membrane composition which secures high encapsulation efficiency of hemoglobin and exhibits excellent physical stability and in-vivo stability; and a method for producing the hemoglobin-containing liposome. A hemoglobin-containing liposome includes a hemoglobin solution as an internal fluid of a liposome, in which the membrane of the liposome is constituted of a lipid mixture of a phospholipid, cholesterol, and a saturated higher fatty acid, and a molar ratio of the cholesterol to the phospholipid (cholesterol/phospholipid) is 0.7 to 1.0, and the content of stearic acid in the lipid mixture is 25 to 30 mol %. Preferably, the membrane of the liposome further contains a polyethylene glycol-bound phospholipid in an amount of 0.8 mol % or more relative to the total amount of the lipids constituting the membrane, and the polyethylene glycol-bound phospholipid is bound onto the outer surface of the membrane.

This application is a continuation of International Application No. PCT/JP2012/073810 filed on Sep. 18, 2014, and claims priority to Japanese Application No. 2011-213416 filed on Sep. 28, 2011, the entire content of both of which is incorporated herein by reference.

### TECHNICAL FIELD

The present invention generally relates to a hemoglobin-containing liposome which secures high encapsulation efficiency of hemoglobin and exhibits excellent physical stability and in the in-vivo stability; and a method for producing the hemoglobin-containing liposome.

### BACKGROUND DISCUSSION

A method of utilizing hemoglobin derived from red blood cells as a substance responsible for oxygen transport of artificial oxygen carrier has been investigated. It is known that a free hemoglobin lacks the in-vivo stability, and has a toxic effect and thus causes various histological damages. Attempts to use a molecule of hemoglobin that is chemically modified and stabilized also have been continued. However, these problems have not yet been resolved.

On the other hand, a hemoglobin-containing liposome in which hemoglobin is encapsulated in a liposome is a analogical structure of red blood cells in the point that hemoglobin is encapsulated in vesicles, and is considered that the toxic effect of hemoglobin can be avoided, and thus the investigation as an artificial oxygen carrier has been conducted.

As to the hemoglobin-containing liposome, in the past, a composition of membrane components for the encapsulation of hemoglobin into a liposome in high yield has been disclosed in International Application Publication No. WO 2003/015753, and a production method for producing hemoglobin-containing liposome has been disclosed in Japanese Patent Application Publication No. 2005-2055. Investigation of the liposome membrane capable of achieving the conditions of in-vivo stability of liposome while maintaining the high yield of hemoglobin and keeping the purpose for encapsulation of hemoglobin into vesicles, has not been sufficient for the combination of the physical and chemical properties that are the ratio of the components, the average particle size, and the ratio of hemoglobin and lipid.

When the hemoglobin-containing liposome is prepared, in order to encapsulate hemoglobin into a liposome without the denaturation, emulsification is required to be performed under low temperature conditions. A phospholipid that is commonly used as a constituent material of liposome membrane has the high homology with a biological membrane component and the high biocompatibility. In particular, a phospholipid constituted of saturated fatty acids has already applied for medication as a substance that can be used safely. However, in the case of a saturated phospholipid, the phase transition temperature is high, and thus the liposome formation at low temperature range is difficult.

On the other hand, it has been clarified that by the mixture of cholesterol into a saturated phospholipid, the distribution of phase transition temperature is changed, and then by the mixing of a fatty acid, hemoglobin can be encapsulated into a liposome even under the emulsification with condition of low temperature. However, it has been also disclosed, for example in International Application Publication No. WO 2003/015753, that when the amount of fatty acids is excessively increased, the liposome itself is destabilized.

Therefore, it is necessary to properly identify the ratio of the materials in the mixture to balance the hemoglobin yield and the stability of liposome. However, even under the condition that the physical stability of the liposome is sufficiently high, in vivo, by the interaction with the biological components, there may be a possibility of the leakage of hemoglobin. If the hemoglobin liberated or released into the blood is a small amount, the hemoglobin binds to haptoglobin in the blood, is carried to the liver and processed. Therefore, the possibility of having adverse effects on the living body is relatively low. If the amount of the hemoglobin liberated into the blood exceeds the amount that can be processed in the liver, free hemoglobin is present in the blood. The amount of haptoglobin in the blood can cover a considerably wide range, and it is difficult to strictly specify the amount that can be processed, and the hemoglobin concentration in the blood plasma in the case where the mixing amount ratio of the fatty acid (stearic acid) is 35 mol % or more is considered to have a possibility that free hemoglobin exceeds the possible concentration range of the processing. It has been known that when the hemoglobin concentration exceeds the possible concentration range of the processing, the free hemoglobin is present in the blood plasma, the hemoglobin dissociates into a dimer easily, and the dissociated dimers are filtered in the renal tubules and excreted in the urine. However, when the hemoglobin leaks in a large amount, the hemoglobin is accumulated in the renal tubules and causes toxic effects.

Further, it is considered that the hemoglobin leaks to the extravascular from the gaps between the vascular endothelial cells, easily binds to and traps the nitric oxide (NO) that is produced by the vascular endothelial cells and is a factor that regulates the tonus and relaxation of the vascular smooth muscle cells, and causes the contraction of vascular smooth muscle cells. It is also considered that in the artificial oxygen carrier that is a type of the artificial oxygen carrier obtained by the chemical modification of hemoglobin, this phenomenon is involved in the vasoconstriction and the effect on the cardiac muscle, which can become a side effect issue. The capability of avoiding this phenomenon is the most important point of the concept of the encapsulation of hemoglobin, and the leakage of hemoglobin from the capsule may jeopardize the concept itself.

Further, particularly in the case where the content of fatty acids is increased, there becomes a problem that from the characteristics of the fatty acid, the charge of the liposome surface is inclined to the negative side, as a result, the activation of a complement system easily occurs, and an issue arises in that the liposome is destabilized in vivo, the in-vivo life is shortened, or the like.

To encapsulate substances as efficiently as possible into a liposome, the average particle size is increased, and the liposome membrane is formed relatively thin, that is, it is effective to minimize the ratio of lipid to the hemoglobin. On the other hand, increasing the particle size possibly promotes the uptake into the reticuloendothelial system in vivo and shortens the life in the blood. See: Ishida O et al., Size-dependent extravasation and interstitial localization of polyethyleneglycol liposomes in solid tumor-bearing mice, Int J Pharmacol, 1999; 190: 49-56; and Awasthi V D et al., Circulation and biodistribution profiles of long-circulating PEG-liposomes of various size in rabbits, Int J Pharmacol, 2003; 253: 121-132.

By relatively decreasing the ratio of membrane lipid components to the substances to be incorporated into the internal aqueous phase due to the increase of the particle size, there is also a safety advantage in that it can reduce the load of lipid of liposome membrane into a living body when the active components, which are present in the internal aqueous phase, that is, correspond to the hemoglobin in the hemoglobin-containing liposome, are administered in vivo. On the other hand, there is also the problem that in a liposome having the composition of a lipid membrane component having the temperature characteristics that allow the emulsification at low temperature range, the larger the particle size, and the relatively thinner the membrane, the easier the liposome itself becomes physically vulnerable and unstable, and particularly in vivo, the easier the leakage of hemoglobin occurs.

Further, as to a liposome, it has been known (See Japanese Application Publication No. 2-149515) that by incorporating a hydrophilic polymer structure such as a polyethylene glycol-bound phospholipid onto the surface of liposome membrane, the in-vivo stability can be improved. In addition, as to a hemoglobin-containing liposome, in the same way, as a measure to avoid the aggregation of liposomes in the blood plasma or the biological reaction caused by the administration of the liposome, a method of modifying the surface of liposome membrane by a polyethylene glycol-bound phospholipid has been disclosed. However, the investigation has never been conducted in consideration of the requirements to achieve both high yield and in-vivo stability.

As an effective measure to prevent the complement system activation or the promotion of the uptake into the reticuloendothelial system by the above-described liposome, a method of modifying the membrane of liposome by a hydrophilic polymer such as polyethylene glycol has been known, and a method of modifying only the surface of liposome membrane by a polyethylene glycol-bound phospholipid has been disclosed (See Bradley A J et al., Inhibition of liposome-induced complement activation by incorporated poly(ethylene glycol)-lipids, Arch Biochem Biophys, 1998; 357(2): 185-94). However, in order to achieve the above-described high yield, in the hemoglobin-containing liposome in which the mixing amount of fatty acids has been increased, the conditions required for the neutralization state of the negative charge on the membrane surface, in which the activation of the complement system hardly occurs, have not been known.

Further, as to the amount to be incorporated onto the surface of liposome membrane of a polyethylene glycol-bound phospholipid, the incorporation amount into the membrane is also increased depending on the amount added. However, it is not intended that the addition amount may be simply increased, as the excessive addition to increases the ratio of the polyethylene glycol-bound phospholipid in a free form. A polyethylene glycol-bound phospholipid itself is a substance having a surface-active effect due to the amphipathic nature. There is a report that in the case where the polyethylene glycol-bound phospholipid is present in a free form in a high concentration, the leakage of the encapsulated substances from the liposome is resulted (See Kasbauer M et al., Polymer induced fusion and leakage of small unilamellar phospholipid vesicles: effect of surface grafted polyethylene-glyucol in the presence of free PEG, Chem Phys Lipids, 1997; 86(2): 153-159). Further, there is also a report showing the possibility that the amount of the polyethylene glycol-bound phospholipid incorporated into the liposome membrane affects the leakage of the encapsulated substances (See Hshizaki K et al., Effects of poly(ethylen glycol) (PEG) chain length of PEG-lipid on the permeability of liposomal bilayer membranes, Chem Parm Bull, 2003; 51(7): 815-820). In addition, there is further a problem that when the addition amount of the polyethylene glycol-bound phospholipid is increased, during the incorporation process of the polyethylene glycol-bound phospholipid in the production process, the liposome membrane is easily destabilized.

### SUMMARY

(1) As a constituent of the lipid membrane of liposome, the combination of a phospholipid and cholesterol is commonly used, cholesterol has characteristics that for an unsaturated fatty acid phospholipid, the permeability of the membrane and the fluidity are decreased, and the liposome membrane is stabilized, on the other hand, for a saturated fatty acid phospholipid, cholesterol is considered to abolish the phase transition and to enhance the fluidity of the membrane. These characteristics are considered that during the emulsification of a protein such as hemoglobin in low temperature range, the encapsulated substances are easily incorporated into a liposome, and the encapsulation efficiency of the incorporated substance is improved. That is, particularly, it is advantageous for the encapsulation of the heat-sensitive substances in low temperature range. In fact, there are disclosures of a method of incorporating hemoglobin into a liposome at a temperature below the phase transition temperature of the membrane component substance by the addition of cholesterol for a phospholipid (Japanese Patent Publication (JP-B) No. H05-64926), and of a hemoglobin-containing liposome and the production method thereof, in which the cholesterol at a weight ratio of 10 to 50% (at a molar ratio of 21 to 100%) is added into a phospholipid, and the liposomal is performed (Japanese Application Publication (JP-A) No. H02-295917). However, an investigation on the relationship between the mixing amount of cholesterol, and the yield of hemoglobin has not been sufficiently conducted.

(2) Further, during the liposome formation, a phenomenon such as aggregation or fusion of the liposomes easily occurs, and thus becomes a destabilizing factor in the preparation of liposome. On the other hand, there is a disclosure that by the addition of a substance having a negative charge as a constituent of the liposome membrane, the phenomenon can be suppressed due to the electrostatic repulsion (Japanese Application Publication (JP-A) No. H01-180245). In addition, it has also been shown that in the case where a fatty acid is used as a negative charge substance, as in the case of cholesterol, when the fatty acid at a ratio of a certain level or more is added to the phospholipid or the cholesterol, the incorporation efficiency of a substance into a liposome is remarkably improved, on the other hand, it has been further disclosed that when the amount of fatty acids is increased excessively, the liposome membrane is destabilized, and the leakage of the encapsulated hemoglobin is increased, therefore, there is the optimal range (See International Application Publication No. WO 2003/015753). However, the information disclosed herein on the stability of a liposome is only information related to the physical stability in vitro, and when a liposome is used for medication, an investigation on the in-vivo stability that is essentially important in view of the safety and the efficacy has not been sufficiently conducted.

(3) A liposome is a vesicle that is clad in a lipid bilayer membrane, and the volume ratio of the internal aqueous phase of the vesicle that is clad in a single layer (unilamellar) is larger than that of the vesicle that is clad in a multiple layer (multilamella), and thus the encapsulation efficiency of the encapsulated substances per unit amount of lipid becomes higher. Further, if the thickness is the same, a liposome having larger particle size has relatively higher voidage of the internal aqueous phase to the lipid membrane, and can be an efficient carrier of encapsulated substances. On the other hand, as to a liposome having a small number of membranes, or a liposome having large particle size and relatively thin thickness, the leakage of the encapsulated substances from a liposome can rather easily occur. In addition, it has been known that in the state in which the particle size of a liposome exceeds 250 to 300 nm, the uptake into the reticuloendothelial system is drastically increased (Klibanov A L et al., Activity of amphipathic poly(ethylene glycol) 5000 to prolong the circulation time of liposomes depends on the liposome size and is unfavorable for immunoliposome binding to target, Biochem Biophys Acta, 1991; 1062: 142-148; Litzinger D C et al., Effect of liposome size on the circulation time and intraorgan distribution of amphipathic poly(ethylene glycol)-conjugating liposomes, Biochem Biophys Acta, 1994; 1190: 99-107). From the point of view of the in-vivo stability, the particle size of the liposome is required to be suppressed below the level described above.

Further, it has been found that there is a correlation between the weight of hemoglobin and lipid (hemoglobin/lipid) and the average particle size, the theoretical yield of hemoglobin that is calculated from the charged amount of hemoglobin and lipid in the case where the average particle size is 200 nm is decreased to around 70% for that in the case where the average particle size is 250 nm.

(4) As described above, the addition of fatty acid results in giving a charge to the liposome membrane, and contributes to the prevention of the aggregation of liposomes in the production process. On the other hand, in the case where the charge of the liposome membrane is inclined to the negative side, it is considered that the activation of a complement system easily occurs, and the liposome is destabilized in vivo, or by a foreign-body reaction, the uptake by the reticuloendothelial system is enhanced. Further, in vivo, there is an exposure of the binding of a protein in the blood, or the uptake into the foreign substance processing cells or organs, and there is a disclosure that due to the modification of the surface of liposome membrane by a hydrophilic polymer substance such as a polyethylene glycol-bound phospholipid, the aggregation of liposomes in vivo, and the processing for a foreign substance are suppressed, and the in-vivo stability is improved (See Japanese Patent Publication (JP-B) No. H07-20857, Japanese Application Publication (JP-A) No. H04-5242, and Japanese Application Publication (JP-A) No. H03-218309).

However, a detailed investigation to find out the optimal range of the modification conditions of PEG phospholipid from the relationship between the surface charge of the liposome and the biological reaction has not been sufficiently conducted. Further, it is not intended that the PEG phospholipid may be added in a large amount, as it has been assumed that the excessive addition increases the PEG phospholipid in a free form, and as a result, due to the surface-active effect, the destabilization of the liposome may be generated. Actually, the amount added, with which the amount does not become excessive, and the modifying effect is sufficiently exerted, has not been clarified.

The present inventors investigated item (1) described above. As a lipid constituting the liposome membrane as disclosed here by way of example, a natural or synthetic lipid can be used, and as a phospholipid, particularly, a hydrogenated phospholipid is suitably used. It has been found that in the case where cholesterol in an amount in the vicinity of the equimolar ratio is added to the phospholipid, the yield of hemoglobin and membrane lipid components becomes satisfactory, and in the case where cholesterol in an amount at the equimolar ratio or more is added, rather, the yield of hemoglobin and lipids is decreased during the preparation of a liposome.

Further, as to item (2) described above, particularly, in the case of a hemoglobin-containing liposome, the advantage of the liposomal, that is, the advantage of using the hemoglobin encapsulated in the vesicle of a lipid is to prevent the toxicity caused by hemoglobin and the adverse biological reactions, which are caused when the hemoglobin is present in a free form. Therefore, easy occurrence of the leakage of hemoglobin in vivo jeopardizes the basic concept itself for the encapsulation, and the possibility of the occurrence of the problem in terms of the safety is increased. Thus, intensive investigations were carried out on the amount of fatty acids addition, with the degree of incorporation of hemoglobin (encapsulation efficiency), and the leakage of hemoglobin in vivo as an index, the optimal molar ratio for the total amount (the total number of moles) of lipid constituting the liposome membrane, which is obtained by the combination of a phospholipid, cholesterol, and a fatty acid, and it was discovered that the amount of fatty acid at a molar ratio of 25 to 30% for the total amount of lipids is appropriate as the condition capable of satisfying both requirements described above. At this time, as the fatty acid, a saturated higher fatty acid is suitably used, in particular, in the case where a phospholipid of acyl chain length C18 is used as the phospholipid, the stearic acid in which the number of the carbon atoms is equal, is suitably used.

As a result of the investigation on item (3) above, average particle size and the ratio of hemoglobin to lipid, described above, without significantly impairing the in-vivo stability, while the incorporation of hemoglobin into a liposome is performed as efficiently as possible, the leakage of hemoglobin is suppressed, and the in-vivo stability is secured, it has been found that the average particle size is at least 200 nm or more, on the other hand the average particle size is set in the range not exceeding 250 nm, the preparation of liposome is performed so that the weight ratio of the hemoglobin to the lipid (hemoglobin/lipid) can be in the range of 1.0 to 2.0 and preferably 1.1 to 1.6, and thus the object can be achieved.

In addition, as to the addition amount of a polyethylene glycol-bound phospholipid, regarding the liposome preparation of the hemoglobin-containing liposome, an investigation was conducted on the modification conditions of PEG phospholipid that neutralizes the surface charge and minimizes the complement system activation. It has been found that as the limit amount not increasing the free PEG phospholipid, which satisfies the conditions described above, the amount of PEG phospholipid is 0.8 to 1.1 mol % by a molar ratio for the total amount of the lipids constituting the membrane.

From the above, the following disclosure is provided.

The disclosure here provides a hemoglobin-containing liposome, containing a hemoglobin solution as an internal fluid of a liposome, in which the membrane of the liposome encapsulating the hemoglobin solution is constituted of a lipid mixture of a phospholipid, cholesterol, and a saturated higher fatty acid, and a molar ratio of the cholesterol to the phospholipid (cholesterol/phospholipid) is 0.7 to 1.0, the content of stearic acid in the lipid mixture is 25 to 30 mol %.

The average particle size of the above-described hemoglobin-containing liposome is preferably 200 to 250 nm.

The ratio (mass ratio) of hemoglobin to the lipid mixture (hemoglobin/the lipid mixture) is 1.0 to 2.0, and preferably 1.1 to 1.6.

The embodiment of the hemoglobin-containing liposome disclosed by way of example here is preferably that the membrane of the liposome further contains a polyethylene glycol-bound phospholipid in an amount of 0.8 mol % or more relative to the total amount of the lipids constituting the membrane, and the polyethylene glycol-bound phospholipid is bound onto the outer surface of the membrane.

The hemoglobin-containing liposome in this embodiment has a zeta potential of 0 mV or more.

Further, in this embodiment, the amount of polyethylene glycol-bound phospholipid is specified to be 0.8 to 1.1 mol % relative to the total amount of the lipids constituting the membrane.

Also disclosed is a method for producing a hemoglobin-containing liposome such as described above.

According to the disclosure here representing an example of the method, the hemoglobin-containing liposome as an artificial oxygen carrier can be prepared with a relatively high yield of hemoglobin and suppression of the leakage of hemoglobin in vivo, and which is present stably in the blood and can be used safely.

### BRIEF DESCRIPTION OF DRAWING FIGURES

FIG. 1 is a diagram showing the physical stability of hemoglobin-containing liposome for each composition in a lipid mixture constituting the liposome membrane.

FIG. 2 is a diagram showing the stability of hemoglobin-containing liposome in vivo for each composition in a lipid mixture constituting the liposome membrane.

FIG. 3 is a diagram showing the relationship between the average particle size of hemoglobin-containing liposome and the ratio of the hemoglobin to the lipid (hemoglobin/lipid).

FIG. 4 is a diagram showing the relationship between the addition amount of PEG into a hemoglobin-containing liposome and the incorporation amount.

FIG. 5 is a diagram showing the relationship between the addition amount of PEG into a hemoglobin-containing liposome and the zeta potential.

FIG. 6 is a diagram showing the relationship between the complement system activation in the human blood plasma by a hemoglobin-containing liposome and the zeta potential.

### DETAILED DESCRIPTION

A liposome is composed of a phospholipid bilayer membrane, and is an aqueous dispersion of a closed vesicle (liposome capsule) having a structure that defines a space separated from the outside by the membrane generated on the basis of the polarity of a hydrophobic group and a hydrophilic group of a lipid. The aqueous phases inside and outside the closed vesicle, across the membrane, are referred to as an internal fluid, and an external fluid, respectively. A hemoglobin-containing liposome is a liposome preparation that is a liposome capsule in which hemoglobin is incorporated, that is, as the internal fluid, a hemoglobin solution is encapsulated.

In the disclosure here, representing an example of the disclosed liposome, the membrane of the liposome (liposome membrane) is constituted of a lipid mixture of a phospholipid, cholesterol, and a saturated higher fatty acid.

The phospholipid is a main component of the biological membrane, and is an amphiphile having a group of a hydrophobic group constituted of a long-chain alkyl group and a hydrophilic group constituted of a phosphate group, in the molecule. The phospholipid can be used in any form of natural phospholipid or synthetic phospholipid, as long as it can form a liposome having the structure described above. Examples of the phospholipid may include phosphatidylcholine (may also be referred to as lecithin), phosphatidylethanolamine (abbreviated PE), phosphatidic acid, phosphatidylserine, phosphatidylinositol, phosphatidylglycerol, further a sphingophospholipid such as sphingomyelin, a natural or synthetic phospholipid such as cardiolipin or a derivative thereof, and a derivative bound to a saccharide (glycolipid) and a hydrogenated product (saturated phospholipid) thereof.

Among them, a saturated phospholipid is preferable. Specific examples of the saturated phospholipid include phosphatidylcholine, phosphatidylethanolamine, phosphatidic acid, phosphatidylserine, phosphatidylinositol, phosphatidylglycerol, a hydrogenated product such as sphingomyelin, and a mixture thereof. In particular, a saturated phospholipid derived from an egg yolk or a soybean, and having a hydrogenation rate of 50% or more is preferred.

In the disclosed example here, cholesterol is present in an amount of 0.7 to 1.0 mol relative to 1 mol of the phospholipid described above.

Examples of the saturated higher fatty acid include a saturated higher fatty acid having a linear chain of 12 to 18 carbon atoms, and specific examples of the saturated higher fatty acid include lauric acid, myristic acid, palmitic acid, and stearic acid. Particularly, stearic acid is preferable. In the disclosure here, the content of the saturated higher fatty acid is 25 to 30 mol % relative to the total amount of the lipid mixture, that is, the phospholipid, the cholesterol, and the saturated higher fatty acid.

As described above, particularly in a liposome encapsulating hemoglobin, the membrane of the liposome is constituted of a limited composition of lipid mixture in which a molar ratio of the cholesterol to the phospholipid (cholesterol/phospholipid) is 0.7 to 1.0, and the content of the saturated higher fatty acid is 25 to 30 mol %. Thus, while the hemoglobin yield and lipid yield, and the high encapsulation efficiency of hemoglobin (a ratio of hemoglobin to lipid, hemoglobin/lipid) during the production are secured, the strength of liposome membrane is maintained even though the hemoglobin is contained as the internal fluid in a high concentration. As a result, the physical stability, and also the liposome (membrane) stability that hardly occurs, the internal fluid leakage and the like at the time of in-vivo administration can be obtained.

In the example disclosed here, the preferred embodiment is that the membrane of the liposome is modified with a polyethylene glycol (PEG)-bound phospholipid. The molecular weight of PEG is not particularly limited, however, usually the average molecular weight Mw is around 500 to 10,000. Examples of the phospholipid of PEG-bound phospholipid may also include a phospholipid similar to the liposome membrane component described above, and is not particularly limited, however, examples of the PEG-bound phospholipid include typically, readily available polyethylene glycol-bound distearoylphosphatidylethanolamine (PEG-DSPE).

In the example disclosed here, the PEG-bound phospholipid is contained in an amount of 0.8 mol % or more relative to the total amount of the lipids constituting the membrane.

In the case of the liposome disclosed here by way of example, the PEG-bound phospholipid is bound onto the outer surface of the liposome membrane. In the case where only the outer surface of liposome membrane is modified with a PEG-bound phospholipid, the liposome becomes a structure in which a PEG chain is extended only from the outer surface of the membrane of liposome (capsule) to the side of external fluid.

In addition, surface modification by a PEG-bound phospholipid exerts a protein adsorption suppressing effect on a liposome surface at the time of in-vivo administration, and also exerts an aggregation preventing effect in the blood plasma of liposome, and an effect of causing prolonged retention in the blood.

In the disclosure here, in addition to these known effects, particularly in order to make the surface potential of liposome neutral or positive, a PEG-bound phospholipid is incorporated in a larger amount than the usual amount specified above. In the case where the amount of incorporation of the PEG-bound phospholipid is 0.8 mol % or more relative to the total amount of the lipids constituting the membrane, the zeta potential of liposome preparation becomes 0 mV or more, that is, the surface potential of liposome becomes neutral or positive. In this case, even in the liposome preparation that contains a large amount of fatty acids as the charged substance, the strength of liposome membrane is maintained in vivo, and further the activation of a complement system in vivo can be avoided.

Further, the upper limit of the amount of the PEG-bound phospholipid described above, as will be described in Production Examples below, from the point of view of the production efficiency that lowers the incorporation efficiency even the PEG-bound phospholipid is used in a large amount, the amount of the PEG-bound phospholipid is preferably 1.1 mol % relative to the total amount of the lipids constituting the membrane.

The hemoglobin-containing liposome can be prepared by a common procedure that prepares a liposome (dispersion) from a membrane component containing a phospholipid, with the use of a lipid mixture specified in the above as a membrane component, and by the incorporation of a hemoglobin solution as an internal fluid.

The hemoglobin solution can be prepared in accordance with the method described, for example, in paragraphs [0032] to [0038] of Japanese Application Publication No. 2006-104069, and with the reference to the description, a detailed explanation is omitted as it is disclosed in the noted published application.

In the case where the raw material of hemoglobin is natural blood, the red blood cell membrane (stroma) is destroyed, that is, hemolyzed, then the red blood cell cytosol such as the stroma, and the blood group substance is separated and removed to obtain stroma-free hemoglobin (SFH), and then processing such as purification, and concentration is performed to prepare a stroma-free hemoglobin solution that is appropriate for the liposome preparation, and is safe and possesses high purity.

The sterility of a naturally-derived hemoglobin solution is assured by the application of a known filtration method, and further the safety of a naturally-derived hemoglobin solution is assured by the virus removal and inactivation. A known method can be used for the removal or inactivation of virus, as long as the method does not substantially denature the hemoglobin protein. For example, there are a virus removal treatment by an ultrafiltration membrane or a virus removal membrane, a heat treatment, a short time heat treatment by microwave irradiation, an ultraviolet irradiation treatment, a treatment using a photosensitizing action that uses a photosensitizing substance such as dimethyl methylene blue, and an inactivation treatment such as a SD (solvent detergent) method. More specifically, a virus inactivation treatment by the heating of a hemoglobin solution at 65° C. or more for 10 hours or by a solvent detergent method, and a virus removal treatment by an ultrafiltration membrane having a fractionation molecular weight of around 100,000 to 300,000 or by a virus removal membrane (Planova manufactured by Asahi Kasei Corporation, Viresolve manufactured by Merck Millipore, and the like) are preferably performed.

The hemoglobin solution after the purification is desirably incorporated into a liposome capsule usually at a concentration of 40 to 50%. In order to obtain this concentration, the concentration can be performed by ultrafiltration concentration using an ultrafiltration filter having a cut-off molecular weight of around 30,000, or the like.

Further, the hemoglobin solution can contain a substance for the purpose of suppressing the oxidation of hemoglobin. In addition, a phosphate compound such as 2,3-diphosphoglycerate (2,3-DPG), pyridoxal phosphate, and inositol hexaphosphate (IP6) may be added as an allosteric effector.

The incorporation of the hemoglobin solution into a liposome capsule can be performed by a common procedure, for example, by the hydration of the lipid mixture of membrane component, and then by agitation in a high speed stirrer with the hemoglobin solution, a suspension in which liposome capsules are dispersed can be obtained. For this suspension, centrifugation or membrane filtration treatment is performed to remove the hemoglobin solution that was not incorporated into a liposome, and then the hemoglobin-containing liposome dispersion is obtained by using an isotonic solution such as a saline solution as the external fluid.

The average particle size of the hemoglobin-containing liposome is preferably smaller than that of red blood cells. Generally, the average particle size is adjusted to 200 to 250 nm by filter treatment.

In addition, using a circulation filtration system by a ultrafiltration of a fractionation molecular weight of 300,000, in a concentration operation with addition of a saline, the hemoglobin that was not incorporated into a liposome is removed, and further the intended concentration can be obtained.

In the disclosure here, it has been confirmed that by a liposome membrane of the lipid mixture specified above, in such a hemoglobin-containing liposome having an average particle size of 200 to 250 nm, the mass ratio of the hemoglobin to the lipid (hemoglobin/lipid) can be 1.1 to 1.6.

As described above, after the preparation of the hemoglobin-containing liposome, by the addition of PEG-bound phospholipid in an amount corresponding to the specific amount described above, a hemoglobin-containing liposome that is a preferred embodiment disclosed here and the outer surface of which is modified with a PEG-bound phospholipid can be obtained.

### EXAMPLES

Hereinafter, Examples in accordance with the disclosure here will be described. These Examples are for the purpose of explaining or illustrating the disclosure here by way of example, but the scope of the present invention should not be construed to be limited by the description of these Examples.

The materials used will be shown in the following.

A hemoglobin solution (hemoglobin concentration is 40 w/v % or more): red blood cells were hemolyzed, extracted, and purified, from a human packed red blood cell preparation, and then inositol hexaphosphate (IP6) was added in an equimolar amount to hemoglobin (Hb), and thus the hemoglobin solution was prepared.

Hydrogenated phosphatidylcholine (HSPC): (Lipoid KG)

Cholesterol: (Solvay pharmaceuticals B.V.)

Stearic acid: (Nippon Fine Chemical Co.,Ltd.)

Polyethylene glycol-bound phospholipid: PEG₅₀₀₀-DSPE (polyethylene glycol-distearoylphosphatidylethanolamine, the average molecular weight (Mw) of PEG is 5000, NOF CORPORATION)

### Production Example 1

### (1) Preparation of Lipid Mixture

HSPC (molecular weight 790), cholesterol (molecular weight 387), and stearic acid (molecular weight 284) were weighed respectively in the amount shown in Table 1, and heated and dissolved into a predetermined amount of t-BuOH, then the t-BuOH is removed by lyophilization, and thus lipid mixtures (1) to (6) having the predetermined mixing ratio (molar ratio) shown in Table 1 were prepared.

### (2) Preparation of Hemoglobin-Containing Liposome

Into around 77 g of each of the lipid mixtures described above, around 77 mL of water for injection was added respectively, and the lipid was heated and swelled. Into the resultant, around 550 g of hemoglobin solution was added, and mixed thoroughly, then using a high-speed stirring type device, while the mixture was cooled, an emulsion was prepared by the intermittent emulsification in the range of 10 to 45° C.

The emulsion was diluted with a saline solution, and filtered. That is, by using a cross-flow filter having a pore size of 0.45 μm, and a dead-end filter having the same pore size as that above, the coarse particles were removed, and thus the average particle size was controlled in an appropriate range. Further, using a circulation filtration system by a ultrafiltration of a fractionation molecular weight of 300,000, in a concentration operation with addition of a saline, the hemoglobin and IP6 that were not incorporated into a liposome is removed, and concentrated, and thus a saline suspension of hemoglobin-containing liposome (the internal fluid is a hemoglobin solution, and the external fluid is a saline solution) was obtained.

### (3) Surface Modification

Next, into the suspension, finally, PEG₅₀₀₀-DSPE in the required amount that was calculated so that the hemoglobin concentration was 6 w/v % and the PEG₅₀₀₀-DSPE concentration was 0.15 w/v % was added, and the resultant was heated, then PEG₅₀₀₀-DSPE was incorporated into the outer surface of liposome membrane, and thus a hemoglobin-containing liposome (hereinafter, also referred to as a preparation) was obtained.

### <Analysis>

Physical and chemical property values of each of the preparations prepared in the above are shown in Table 2. Further, the measurement methods are shown in the following.

### [Measurement of Average Particle Size]

The preparation sample was diluted with a saline solution, and the average particle diameter of liposome was measured by a light diffraction scattering particle size distribution analyzer (Beckman Coulter LS230).

### [Analysis of Hemoglobin Concentration by Cyanmethemoglobin Method]

Into the preparation sample, a chromogenic reagent solution for a cyanmethemoglobin method was added, the resultant mixture was rapidly cooled and once frozen in liquid nitrogen, then thawed in running water, then into the resultant mixture, a predetermined amount of water was added, and further, under ice-cooling, dimethyl sulfoxide was added, shaken to be mixed, and left to stand, then into which water was added to accurately adjust the volume. The sample solution was thus obtained.

Separately, into a hemoglobin solution at a predetermined concentration of various types, a chromogenic reagent solution was added, then into the resultant mixture, dimethyl sulfoxide was added, and shaken to be mixed, and thus a standard solution was prepared.

A predetermined diluted solution of a chromogenic reagent solution was used as a control, each absorbance of the sample solution and the standard solution was measured at a wavelength of 540 nm, and from the absorbance ratio of the sample solution to the standard solution, the hemoglobin concentration of the sample was calculated.

As to the preparations (2) to (5), the hemoglobin concentration remaining in the external fluid (hemoglobin concentration in external fluid) was measured. As the sample solution of the external fluid, the supernatant that was obtained by the ultracentrifugation (50,000×g×120 minutes) of the preparation was used.

### [Analysis of Membrane Composition]

Into the preparation sample, a predetermined amount of an internal standard solution, further, chloroform were added and shaken vigorously to be mixed, and then the resultant mixture was centrifuged (3,000 rpm×10 minutes). The supernatant was filtered through a membrane filter of 0.20 μm, and used as a sample solution.

Separately, each standard product of HSPC, cholesterol, stearic acid, and PEG₅₀₀₀-DSPE was precisely weighed, then into each standard product, chloroform was added and dissolved, further, into the resultant mixture, an internal standard solution and a predetermined amount of saline solution were added, and thus the resultant mixture was used as a standard solution.

As to the sample solution and the standard solution, reversed-phase HPLC was performed using sodium acetate/acetic acid as a mobile phase, and from the ratio of the peak area of each component to the peak area of the internal standard substance in the sample solution, detected by a differential refractometer, the amount of each component was calculated.

Further, the composition (mol %) of stearic acid, which was obtained by the analysis described above, is a ratio to the total amount of the lipids constituting the membrane of HSPC, cholesterol, and stearic acid, and the composition (mol %) of PEG₅₀₀₀-DSPE is also a ratio to the total amount of the lipids constituting the membrane as well.

### [Hemoglobin Yield]

The hemoglobin amount in the preparation obtained by the method of the Production Example was divided by the hemoglobin amount in the treatment solution before the liposomal treatment, then the obtained value was multiplied by 100, and thus the resultant value was set to the hemoglobin yield.

### [Lipid Yield]

The lipid amount in the preparation obtained by the method of the Production Example was divided by the lipid amount in the treatment solution before the liposomal treatment, then the obtained value was multiplied by 100, and thus the resultant value was set to the lipid yield.

### [Hemoglobin/Lipid (Mass Ratio)]

The hemoglobin concentration was divided by the lipid concentration in the preparation obtained by the method of Production Example, and the resultant value was set to the hemoglobin/lipid.

TABLE 1
Charged amount of lipid raw material

| | | Preparation | | | | | |
|---|---|---|---|---|---|---|---|
| | | (1) | (2) | (3) | (4) | (5) | (6) |
| Prescription ratio (molar | | 1/0.7/1 | 1/1/0.3 | 1/1/0.7 | 1/1/1 | 1/1/1.4 | 1/1.3/1 |
| ratio) of HSPC/cholesterol/stearic | | | | | | | |
| acid | | | | | | | |
| Weighed | HSPC (g) | 117.29 | 125.88 | 114.80 | 325.37 | 100.14 | 100.85 |
| value | Cholesterol | 40.77 | 61.27 | 55.88 | 158.40 | 48.76 | 63.11 |
| | (g) | | | | | | |
| | Stearic acid | 41.91 | 12.85 | 29.31 | 116.23 | 51.12 | 36.03 |
| | (g) | | | | | | |

TABLE 2
Test results

| Preparation | | | | | |
|---|---|---|---|---|---|
| (1) | (2) | (3) | (4) | (5) | (6) |

Analysis value

| | | | | | | | |
|---|---|---|---|---|---|---|---|
| Lipid constituting | HSPC (w/v %) | 2.18 | 2.77 | 2.52 | 2.12 | 1.95 | 2.27 |
| the membrane | Cholesterol (w/v %) | 0.76 | 1.43 | 1.24 | 1.02 | 0.93 | 1.43 |
| | Stearic acid (w/v %) | 0.74 | 0.3 | 0.63 | 0.76 | 0.96 | 0.78 |

| | | | | | | |
|---|---|---|---|---|---|---|
| PEG₅₀₀₀-DSPE (w/v %) | 0.15 | 0.16 | 0.16 | 0.15 | 0.15 | 0.15 |
| Hemoglobin concentration (w/v %) | 6.1 | 6.0 | 6.1 | 5.9 | 6.4 | 6.1 |
| Hemoglobin concentration in external fluid | — | 0.04 | 0.06 | 0.04 | 0.02 | — |
| (w/v %) | | | | | | |
| Average particle size (nm) | 258 | 209 | 214 | 244 | 241 | 216 |

Calculated value

| | | | | | | | |
|---|---|---|---|---|---|---|---|
| Lipid | Cholesterol/HSPC (molar ratio) | 0.7 | 1.1 | 1.0 | 1.0 | 1.0 | 1.3 |
| constituting the | Stearic acid/membrane lipid | 36 | 13 | 26 | 33 | 41 | 29 |
| membrane | (mol %) | | | | | | |

| | | | | | | |
|---|---|---|---|---|---|---|
| PEG₅₀₀₀-DSPE/membrane lipid (mol %) | 0.34 | 0.32 | 0.31 | 0.31 | 0.30 | 0.26 |
| Lipid yield (%) | 56 | 40 | 45 | 56 | 58 | 39 |
| Hemoglobin yield (%) | 31 | 18 | 21 | 28 | 31 | 18 |
| Hemoglobin/lipid (mass ratio) | 1.7 | 1.4 | 1.4 | 1.5 | 1.6 | 1.4 |

As shown in Table 2, it was observed that in the case of the phospholipid/the stearic acid=1/1 (molar ratio) constant, both of the hemoglobin yield and the lipid yield were almost the same as each other when the molar ratio of the cholesterol was 1 or less (preparations (1) and (4)). On the other hand, both the hemoglobin yield and the lipid yield were significantly decreased in the preparation (6) in which the molar ratio of the cholesterol was larger than 1. On the other hand, in the case of the phospholipid/the cholesterol=1/1 (molar ratio) constant (preparations (2) to (5)), these yields were increased as the amount of stearic acid was increased. Further, there was a tendency that the ratio of the hemoglobin to the lipid (hemoglobin/lipid) became smaller as the amount of cholesterol was increased in the case of the phospholipid/the stearic acid=1/1 (molar ratio) constant. On the other hand, in the case of the phospholipid/the cholesterol=1/1 (molar ratio) constant, the ratio of hemoglobin/lipid was increased as the amount of stearic acid was increased.

### Test Example 1

### Hemoglobin Leakage Test Due to Shear Stress

As to the evaluation of the physical stability of liposome, by a method of circulating and passing through a filter, the evaluation was conducted. In this test, as an indicator of the physical strength of liposome, the hemoglobin amount leaked from liposome, which was pressurized by circulating and passing through a membrane filter, was measured as follows.

40 mL of each of the preparations (2) to (5) prepared in the above was put in each plastic container that is used as a reservoir, heated to 37° C., and circulated and passed through a disc filter having a diameter of 26 mm (the pore size is 5.0 μm, the membrane area is 5.3 m², a cellulose acetate membrane manufactured by Sartorius) for 4 hours via a tube attached to a peristaltic pump.

The circulation was terminated and the liquid in the circulation circuit collected, then the liquid in the reservoir was ultracentrifuged (30,000×g×60 minutes), and the liposomes were precipitated, and then the hemoglobin concentration in the centrifuged supernatant was determined by the cyanmethemoglobin method described above.

The value that was obtained by subtracting the value of the hemoglobin concentration in external fluid shown in Table 1 from the quantified value was set to the hemoglobin leakage.

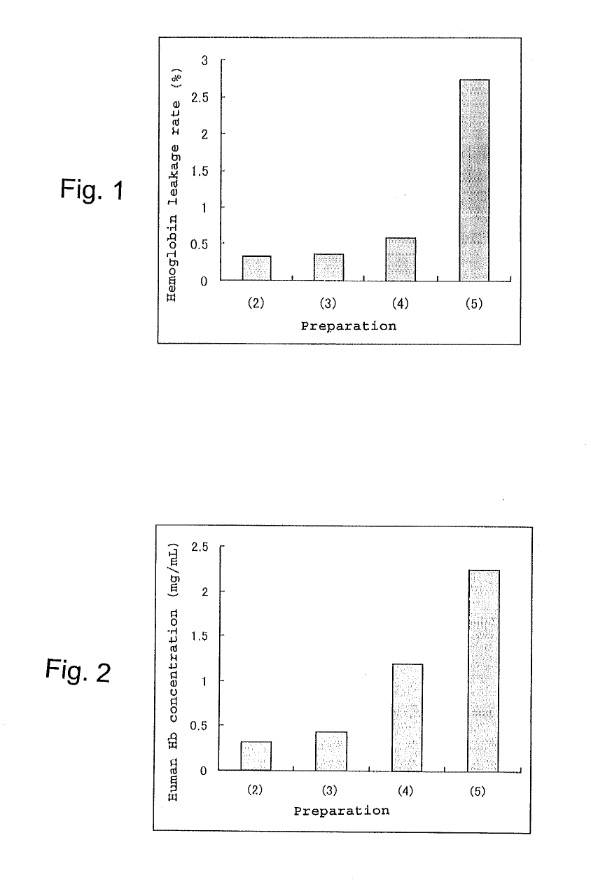

Next, the value that was obtained by subtracting the value of the hemoglobin concentration in the external fluid from the value of hemoglobin concentration in the preparation was set to the hemoglobin concentration in a liposome, and the ratio of the hemoglobin leakage to this concentration was set to the hemoglobin leakage rate. The results are shown in FIG. 1.

### Test Example 2

### Liposome (Membrane) Stability Evaluation In Vivo

Investigation of the concentration of the hemoglobin that had been leaked into rat blood after the intravenous administration of hemoglobin-containing liposome to rats was conducted.

To a Sprague-Dawley rat (SD rat, the body weight was 283.0 to 317.8 g), 20 mL/kg of hemoglobin-containing liposome was administered via the tail vein at a dose rate of 2 mL/kg/minute by using a syringe pump. 5 minutes after the completion of the administration, the rat was subjected to the laparotomy under ether anesthesia, and then the heparinized blood (the final concentration of heparin was 5 to 10 U/mL) was collected from the abdominal aorta. The supernatant obtained by the centrifugation (3,000 rpm×10 minutes) of the whole blood was further ultracentrifuged (50,000×g×120 minutes) to obtain a supernatant, and the hemoglobin concentration in the obtained supernatant was measured.

The human hemoglobin concentration in the sample was obtained by the separated determination using a reversed-phase HPLC gradient method. That is, in the reversed-phase HPLC using the 0.1% aqueous solution of trifluoroacetic acid/0.1% solution of trifluoroacetic acid acetonitrile as the mobile phase, by the authentic preparation of human hemoglobin, a calibration curve was made from the peak area values of the globin that is part of the proteins constituting hemoglobin and the quantified value as the human hemoglobin was calculated from the peak area of the globin of the sample. The results are shown in FIG. 2.

In the Test Example 1 described above, it was observed that the leakage of hemoglobin by physical force was drastically increased in the preparation (5) as compared with the preparation having a content of stearic acid of up to 33 mol % (preparations (2) to (4)) (FIG. 1).

On the other hand, in the liposome stability evaluation in vivo (Test Example 2), the leakage of hemoglobin into the plasma in the preparation having a content of stearic acid of up to 26 mol % (preparations (2) and (3)) was suppressed low, however, the leakage of hemoglobin in the preparation (4) of 33 mol % was increased to 3.5 times that of the preparation (2) of 13 mol %, and in the preparation (5) of 41 mol %, the leakage of hemoglobin was increased to around twice that of the preparation (2) (FIG. 2).

As described above, as to the content ratio of stearic acid, the obtained result was that relative to the total lipid amount of membrane, up to at least around 41 mol %, the larger the content of stearic acid, the better the hemoglobin yield when the liposome was prepared, however, in consideration of the in-vivo stability, the obtained result was that the range up to around 30% was preferable.

### Production Example 2

Each of HSPC (3,149 g), cholesterol (1,543 g), and stearic acid (809 g) was weighed, and heated and dissolved into a predetermined amount of ethanol. Further, the resultant mixture was heated under reduced pressure, the ethanol was removed, and thus a mixture of lipid composed of HSPC, cholesterol, and stearic acid with each ratio of the components was prepared.

In addition, into 4.0 kg of this lipid mixture, 4.0 kg of water for injection was added, the lipid was heated and swelled. Next, into the resultant mixture, 29.5 kg of the hemoglobin solution (the hemoglobin concentration was 40 w/v % or more) that had been obtained by the extraction of hemoglobin from human packed red blood cell preparation, then by the purification of the extracted hemoglobin, and by the addition of an equimolar amount of inositol hexaphosphate into the purified hemoglobin, was added and mixed thoroughly, and thus a mixture of hemoglobin and lipid was obtained.

After that, the mixture of hemoglobin and lipid that was prepared in this ratio was subjected to intermittent stirring emulsification while being cooled, using a high-speed stirring type device in order to control the emulsification temperature in the range of 10 to 45° C. In addition, multiple preparations were prepared by adjusting the stirring conditions during the emulsification.

After the emulsification, into the emulsion, 100 kg of saline solution was added for the dilution, the coarse particles were removed by using a cross-flow filter having a pore size of 0.45 μm, and a dead-end filter having the same pore size as that above, and further, by a ultrafiltration system of a fractionation molecular weight of 300,000, in a concentration operation with addition of a saline, the hemoglobin and IP6 that had not been incorporated into a liposome were removed, and concentrated, and thus a suspension of hemoglobin-containing liposome by a saline solution was obtained. A polyethylene glycol-bound phospholipid was dissolved in a saline solution so that the content of the polyethylene glycol-bound phospholipid could be 0.9 mol % relative to the total amount of the lipids constituting the membrane of the obtained suspension, and the resultant mixture was added into a suspension and subjected to heat treatment.

As to the hemoglobin-containing liposome prepared as described above, in the same manner as in Test Example 1, the hemoglobin amount, the amount of each component of lipids, and the average particle size were measured, and thus the ratio of the hemoglobin to the lipid (hemoglobin/lipid) was calculated.

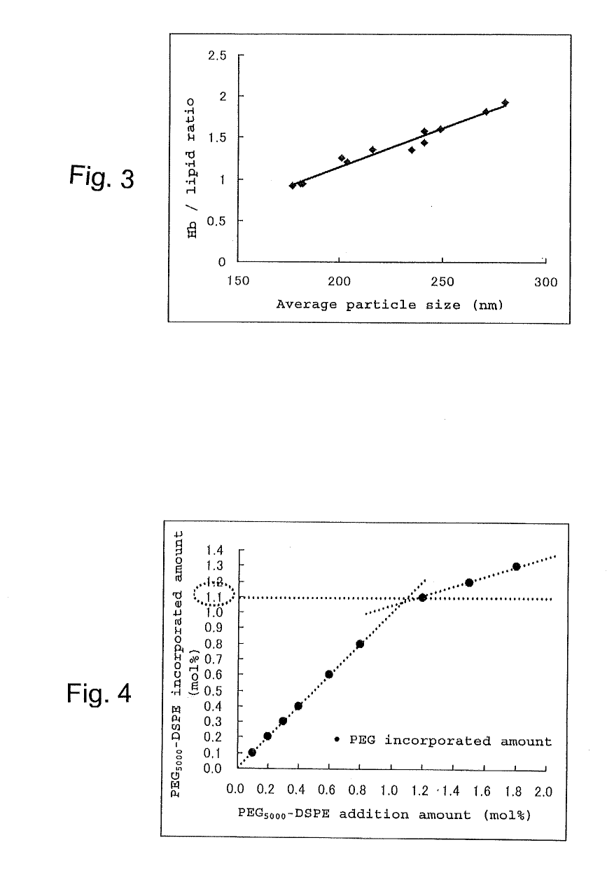

As a result, as shown in FIG. 3, a high correlation was observed between the average particle size and the ratio of the hemoglobin to the lipid (hemoglobin/lipid). That is, in the case where the average particle size of liposomes was in the range of 200 to 250 nm, the ratio of the hemoglobin to the lipid (hemoglobin/lipid) was in the range of 1.1 to 1.6 (FIG. 3).

### Production Example 3

By the method described in Preparation Example 1, a hemoglobin-containing liposome having the same ratio of membrane lipid as that in the preparation (3) was prepared, in the surface modification of the preparation, the addition amount of PEG₅₀₀₀-DSPE was changed in the range of 0.1 to 1.8 (molar ratio) relative to the total amount of the lipids constituting the membrane, and in the same manner as in Preparation Example 1, the liposome surface was modified with the PEG₅₀₀₀-DSPE.

The incorporated amount was measured for each addition amount, and the investigation of the incorporation efficiency of PEG₅₀₀₀-DSPE, the charged state (zeta potential) of liposome, and the complement system activation by the liposome when the liposome was contacted with the plasma, was conducted.

### [PEG Incorporated Amount]

The preparation sample was ultracentrifuged (50,000×g×120 minutes, 10° C.), the supernatant (containing the PEG₅₀₀₀-DSPE that had not been bound) was discarded, and into the remained resultant, a saline solution was added to suspend and to obtain the uniform state.

As to this suspension, in the same manner as in the [Analysis of membrane component] described above, the incorporated amount (mol %) of PEG₅₀₀₀-DSPE was quantified (abbreviated as PEG incorporated amount).

The results are shown in FIG. 4. The PEG incorporated amount was increased with a linear correlation up to the certain level of the addition amount, however, in the region where the addition amount exceeded 1.2 mol %, the gradient became smaller. That is, in the region where the gradient is small, it is meant that as the addition amount is increased, the amount of free PEG-bound phospholipid (PEG₅₀₀₀-DSPE) is increased. It was confirmed that the intersection point of these two straight lines corresponds to the addition amount of 1.1 mol % of PEG₅₀₀₀-DSPE, and the value of this addition amount was the limit of the addition amount, at which the amount of incorporation can be increased without increasing the amount of free PEG₅₀₀₀-DSPE.

### Test Example 3

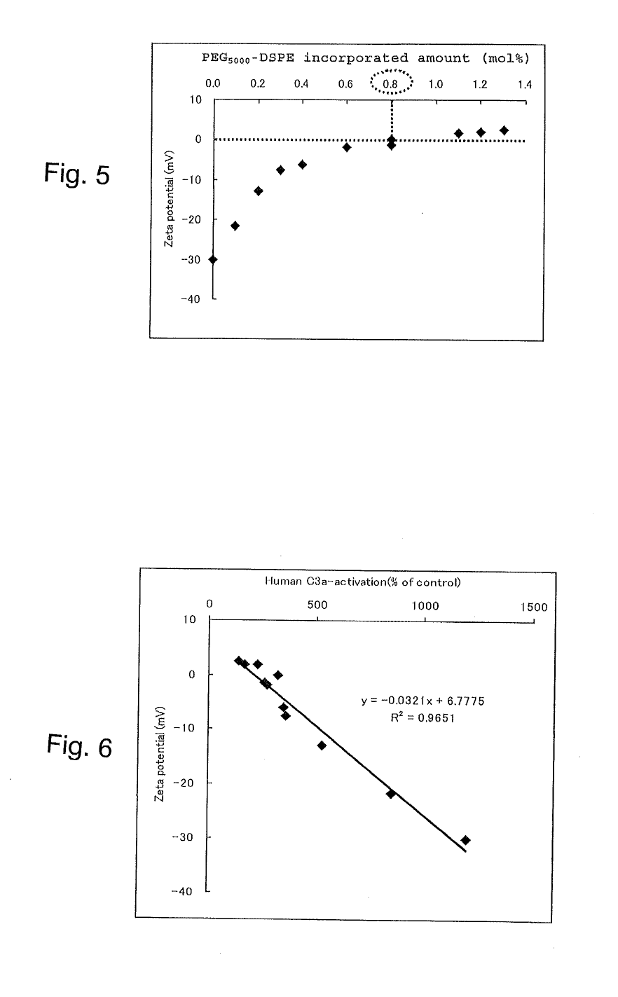

The surface charge of each hemoglobin-containing liposome having a different PEG incorporated amount, which was obtained in Production Example 3, was measured as follows. The results are shown in FIG. 5.

The preparation sample was diluted with phosphate-buffered saline (137 mM of NaCl, 2.7 mM of KCl, and 10 mM of phosphate buffer, pH 7.4) so that the hemoglobin concentration could be 0.06%, and the zeta potential was measured by Zetasizer (Malvern, Zetasizer 3000HS). The results are shown in FIG. 5.

As shown in FIG. 5, the zeta potential was approached the neutral as the PEG incorporated amount was increased, and the zeta potential became almost zero at 0.8 mol % or more of the PEG incorporated amount.

### Test Example 4

As the evaluation indicator of the biological reaction of hemoglobin-containing liposome, the complement system activation in the human plasma was investigated as follows.

The blood (5 U/mL of heparin was added as a final concentration) that was collected from the human median cubital vein was centrifuged (3,000 rpm×15 minutes), then into the obtained centrifuged supernatant (plasma), the hemoglobin-containing liposome in which the PEG incorporated amount was different was mixed at a predetermined ratio, and the resultant mixture was incubated at 37° C. for 30 minutes. After completion of the incubation, the plasma was rapidly cooled on an ice, then into the plasma, a mixed solution of EDTA•Futhan (Torii Pharmaceutical Co., Ltd.) was added as a reaction-stopping solution of the complement system, and the resultant mixture was stored frozen. As to the sample, the C3a concentration was measured by Human C3a ELISA kit.

As to the preparation with each PEG incorporated amount, the C3a concentration for the zeta potential measured in Test Example 3 is shown in FIG. 6.

As shown in FIG. 6, the relationship between the zeta potential and the activation of the complement system showed an inverse correlation, and the activation of the complement system was reduced as the zeta potential approached the neutral.

### Test Example 5

As to the hemoglobin-containing liposome, the effect of the incorporated amount of PEG₅₀₀₀-DSPE on the leakage of hemoglobin in vivo was evaluated.

0.3 mol % and 0.9 mol % of the PEG incorporated amount that was obtained in the same manner as in Production Example 3, each hemoglobin-containing liposome was intravenously administered to rats, and in the same manner as in Test Example 2, the human hemoglobin concentration in the rat blood was measured. The results are shown in Table 3.

In the case of 0.9 mol % of PEG incorporated amount, a result that the leakage of hemoglobin was suppressed was obtained.

TABLE 3

| PEG₅₀₀₀-DSPE incorporated amount | Human Hb concentration (mg/mL) |
|---|---|
| 0.3 mol % | 0.40 ± 0.02 |
| 0.9 mol % | Lower limit of quantitation |
| | (0.30 mg/mL) or less |

The detailed description above describes a hemoglobin-containing liposome representing examples of the medical device disclosed here. The disclosure and the present invention are not limited, however, to the precise embodiments and variations described. Various changes, modifications and equivalents could be effected by one skilled in the art without departing from the spirit and scope of the disclosure as defined in the appended claims. It is expressly intended that all such changes, modifications and equivalents which fall within the scope of the claims are embraced by the claims.

What is claimed is:

1. A hemoglobin-containing liposome, comprising:
a hemoglobin solution as an internal fluid of the liposome;
a liposome membrane encapsulating the hemoglobin solution;
wherein the liposome membrane is comprised of a lipid mixture of a phospholipid, cholesterol, a saturated higher fatty acid, and a polyethylene glycol-bound phospholipid;
a molar ratio of the cholesterol to the phospholipid (cholesterol/phospholipid) is 0.7 to 1.0;
a content of stearic acid in the lipid mixture is 25 to 30 mol %; and
a content of the polyethylene glycol-bound phospholipid is 0.8 to 1.1 mol % relative to a total amount of the lipids constituting the liposome membrane.

2. The hemoglobin-containing liposome according to claim 1, wherein the polyethylene glycol-bound phospholipid in the liposome membrane, is bound to an outer surface of the liposome membrane.

3. The hemoglobin-containing liposome according to claim 2, wherein an average particle size of the hemoglobin-containing liposome is 200 to 250 nm.

4. The hemoglobin-containing liposome according to claim 3, wherein a mass ratio of hemoglobin to the lipid mixture (hemoglobin/the lipid mixture) is 1.1 to 1.6.

5. The hemoglobin-containing liposome according to claim 4, wherein the hemoglobin-containing liposome possesses a zeta potential of 0 mV or more.

6. The hemoglobin-containing liposome according to claim 1, wherein an average particle size of the hemoglobin-containing liposome is 200 to 250 nm.

7. The hemoglobin-containing liposome according to claim 6, wherein a mass ratio of hemoglobin to the lipid mixture (hemoglobin/the lipid mixture) is 1.1 to 1.6.

8. The hemoglobin-containing liposome according to claim 7, wherein the hemoglobin-containing liposome possesses a zeta potential of 0 mV or more.

9. The hemoglobin-containing liposome according to claim 1, wherein a mass ratio of hemoglobin to the lipid mixture (hemoglobin/the lipid mixture) is 1.1 to 1.6.

10. The hemoglobin-containing liposome according to claim 9, wherein the hemoglobin-containing liposome possesses a zeta potential of 0 mV or more.

11. The hemoglobin-containing liposome according to claim 1, wherein the hemoglobin-containing liposome possesses a zeta potential of 0 mV or more.

12. The hemoglobin-containing liposome according to claim 2, wherein the hemoglobin-containing liposome possesses a zeta potential of 0 mV or more.

13. The hemoglobin-containing liposome according to claim 3, wherein the hemoglobin-containing liposome possesses a zeta potential of 0 mV or more.

14. The hemoglobin-containing liposome according to claim 2, wherein a mass ratio of hemoglobin to the lipid mixture (hemoglobin/the lipid mixture) is 1.1 to 1.6.

15. A method for producing the hemoglobin-containing liposome according to claim 1.
